(19) United States
(12) Patent Application Publication
(10) Pub. No.: US 2010/0222707 A1
(43) Pub. Date: Sep. 2, 2010

(54) Carrier for thin-film force sensor for measuring masticatory forces

(75) Inventor: Hans-Werner Weisskricher, Lgel (DE)

Correspondence Address: Dr. Hans-Werner Weisskircher, Schauinsland 31, Igel, 54298, DE

(73) Assignee: Hans-Werner Weisskircher, Igel (DE)

(21) Appl. No.: 12/380,546

(22) Filed: Mar. 2, 2009

(51) Int. Cl. A61C 19/04; A61B 5/22

(52) U.S. Cl. 600/590

(57) ABSTRACT

Measuring isometric bite forces has until now been an impracticable endeavor in clinical practice, since there is no device which is easy to use. Commercially available reusable force sensors were the starting point of the idea to develop a simple device to hold and position these thin-film force sensors while simultaneously protecting them from destructive biting strains. For this reason I developed a bite fork, preferably made of plastic, which can serve as a carrier for these special sensors, and which aid placement on the occlusal surfaces in a repeatable position, without resulting in significant alteration from the habitual bite position.

### BACKGROUND OF THE INVENTION

Being able to record masticatory forces is often of interest to diagnosticians in dentistry or related medical fields.

When measuring bite forces one must differentiate between static forces and dynamic forces.

Recording static bite forces is established while teeth are in contact with an item which does not allow the jaws to move further towards each other. In this condition the masticatory muscles exert isometric forces.

Measuring isometric masticatory forces has the advantage of delivering stable values which make it possible to show a linear correlation between muscle activity and muscle load.

Although the primary application so far is in research, clinical implementation would also be beneficial, but as yet there are no easy to use and inexpensive devices on the market.

The measurement of isometric forces can be achieved in several ways using
strain gauges which are attached at pins mounted in splints which cover parts of the teeth of the upper and lower jaw
special thin-film force sensors which are placed between the teeth

The second method uses sensors very specialized for dental use, but as they are submitted to the enormous shearing forces between the tooth cusps they can only be used only a few times, or only once if intercuspation is unfavorable. Due to these shearing stresses the measurements of the masticatory forces may also be distorted. In addition to these disadvantages, the devices they are attached to and their embedded software are much more expensive than commercially available devices using thin-film force sensors and their software which are mass produced.

The strain gauge systems currently available have the disadvantage of being custom made, and are therefore too expensive and unwieldy for daily clinical use primarily due to the presence of leads. Strain gauge systems are not suited for multiple use, or to be used by different patients. This lack of reusability is caused by the fact that they have to be permanently attached to the measuring device.

### SUMMARY OF THE INVENTION

The objective of my invention was the development of an easy to use and inexpensive device which is able to record isometric masticatory forces bilaterally using thin-film force sensors already available on the market and which allows their reusability.

This is achieved by using an inexpensive plastic fork which can hold commonly available thin-film force sensors firmly solely by clamping them, involves a bite area which protects the force sensitive head of the sensor from destructive shear forces and facilitates reproducible values through the use of positioning aids for the fork itself and for the sensitive head of the force sensor.

### BRIEF DESCRIPTION OF THE DRAWINGS

This invention comprises the device, combinations and arrangements of the parts hereinafter described by way of example and illustrated in the accompanying drawings of a preferred embodiment in which

FIG. 1 shows the top view of the fork without the removable part of the right leg. The dotted lines represent the thin-film force sensor 14.

FIG. 2 shows the side view of the right leg's removable part from the fork's vertical center line perspective.

FIG. 3 shows the removable part of the right leg from the top.

FIG. 4 shows the side view of the removable part from the fork's vertical center line perspective.

### DETAILED DESCRIPTION OF THE INVENTION

My invention consists of a prefabricated wishbone shaped movable fork out of plastic with broadened extensions towards the fork's vertical center line which function as bite area.

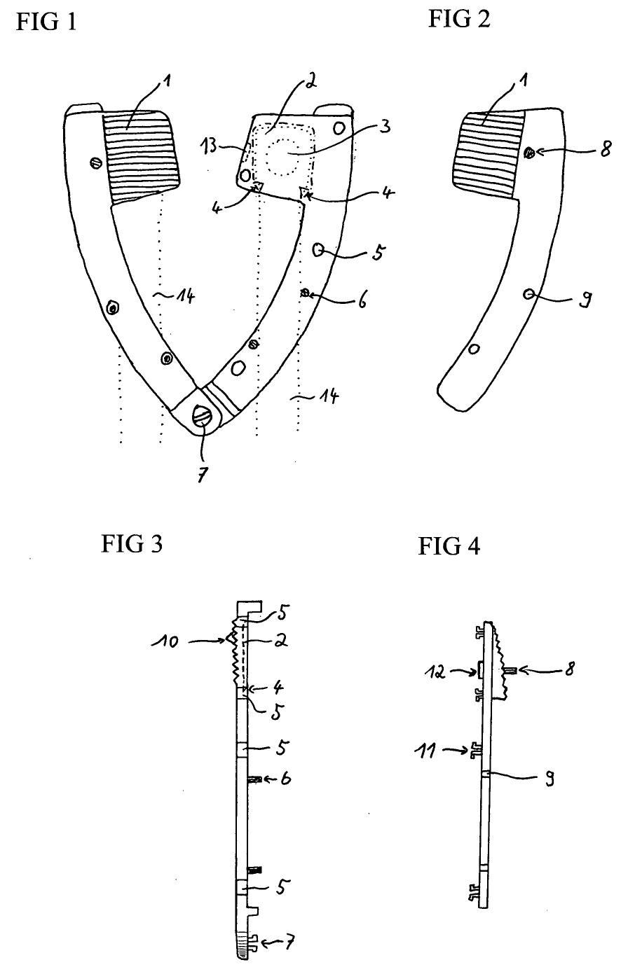

This fork holds two commonly available thin-film force sensors exactly in place. The legs of the fork are split lengthwise, allowing the separated removable part of the leg to clamp the thin-film force sensor when reattached to its base part. As shown in FIG. 1, a deepened area 2 at the bite area 1 is represented by a dashed line. The sensitive part of the head of the thin-film force sensor 3 rests in this cavity without being clamped by the clipped on removable part of the fork. This depression corresponds to the specific sensor head design. An undercut 4 in the wall of the depression allows better retention of the thin-film force sensor. As shown in FIG. 3, at the reverse side of the base part the surface of the bite part is roughened and there is an additional slight ridge 10 to facilitate exact positioning of the lower jaw.

The base parts also have pins for better guidance of the thin-film force sensor. In FIGS. 1 and 3, one can see the two thin-film guiding pins 6 and the four holes 5 which take the four retentive pins of the removable part.

At the hinge of the fork the legs are connected by a screw or a retentive pin 7 so that the angle can be set individually.

As shown in FIG. 2, the removable part is in its shape a smaller copy of the remaining base part including the bite area.

The bite area of the removable part itself may be slanted in order to take into consideration the opening angle of the jaws when the bite area is put between the teeth. The slanted biting area would be roughened through grooves. A fork positioning pin 8 is situated beside the bite area. This pin facilitates reproducibility when recording bite forces in subsequent sessions. The two holes 9 house the thin-film strip guiding pins.

As shown in FIG. 4, at the reverse side of the bite area of the removable part there is a slightly elevated round part 12 which functions as a button to exert pressure on the force sensitive part of the thin-film force sensor. This figure also shows the four retentive pins 11 to clip the removable part onto the base part using corresponding holes 5.

Between the base part and the removable part is a gap 13 which facilitates separation of the two parts with some flat instrument or fingernail.

The surfaces of the biting area of the base and removable part may be roughened to allow better traction during biting and better adherence for impression materials or similar substances.

The force sensors are connected to the PC via an A/D converter before measurement begins. The removable parts of each leg of the bite fork are detached using an appropriate device in the slot at the bite area.

Then the head of the thin-film force sensor is put into the depression of the extension of the leg's base part, the shank of the sensor placed between the guiding pins, and with a slight pull the head part is caught by the undercuts.

Then the removable parts are snapped back onto the base parts of the fork's legs. Now nullification and calibration may be done.

The fork is then placed between the upper and lower jaw so that favorably the positioning pins are located between second premolars and first molars of the upper jaw. The patient is requested to bite together lightly, just enough to hold the bite fork.

In this position an impression material or an acrylic resin can be applied to the biting area from the side to reduce the risk of misleading measurements due to occlusal irregularities.

In an alternative method the materials are put on the biting area prior to placing the bite fork between the teeth.

A further alternative method would involve preparing the bite area in the dental laboratory using casts of the jaws.

The patient bites on the bite areas putting pressure on the sensitive area of the force sensors via the buttons. The sensor and A/D converter transform this pressure into digital values which can be read and submitted to algorithms by the computer.

1. Non-patient-customized bite fork for placing and holding commercially available thin-film force sensors in such a way as to enable the quantitative measuring of bilateral bite forces.

2. Bite fork according to claim 1 further comprising two extensions in which the pressure sensitive heads of the sensor are located, and which serve as bite area.

3. Bite fork according to claim 2 further comprising movable legs that can be adjusted at the hinge by a screw, a retentive pin or by a device holding said movable legs together at the hinge, said legs split lengthwise in such a way as to enable clamping the non-sensitive part of the thin-film force sensors.

4. Bite fork according to claim 3 further comprising pins for positioning the biting area of said bite fork at the teeth and a slanted surface in the said biting area.

5. Bite fork according to claim 4 further comprising in the said biting area a button, thereby serving a definite area of pressure sensing.
